(19) United States
(12) Patent Application Publication
(10) Pub. No.: US 2016/0263312 A1
(43) Pub. Date: Sep. 15, 2016

(54) PORTABLE PUMP AND METHOD FOR PRODUCING SAME

(71) Applicant: SWISSINNOV PRODUCT SARL, Gland (CH)

(72) Inventors: Florent JUNOD, Veigy-Foncenex (FR); Thierry NAVARRO, Gland (CH)

(21) Appl. No.: 15/033,305

(22) PCT Filed: Oct. 30, 2014
(86) PCT No.: PCT/IB2014/002279

§ 371 Date: Apr. 29, 2016

(30) Foreign Application Priority Data
Oct. 31, 2013 (IB) PCT/IB2013/002416

(51) Int. Cl. A61M 5/142; A61M 5/158; A61M 5/145

(52) U.S. Cl. CPC A61M 5/14244; A61M 5/1452; A61M 5/158; A61M 2205/10; A61M 2005/1585

(57) ABSTRACT

The invention relates to a miniature portable pump with mechanical actuation, preferably without a motor, provided with a bowed piston for injecting fluid by manual dosing and/or continuously. The pump consists of a small number of parts with very low production cost, for injecting medication, preferably subcutaneously.

The invention relates to a miniature portable pump with simple mechanical actuation, preferably without a motor, for injecting fluid mainly in the medical field. The system is designed so that it can deliver small, accurate volumes of any type of liquid and medicinal product and of any chemical compound, at variable rates, simply and with low production cost.

### PRIOR ART

There are various types of portable medical subcutaneous infusion pumps as described in U.S. Pat. No. 4,525,164, U.S. Pat. No. 8,137,314 or EP2438938 comprising a curved piston moving within a curved reservoir by means of a motor. These systems are made up of a disposable portion and a reusable portion. The reusable portions are made up of electromechanical elements, including in particular a motor, control electronics and a rechargeable battery. The use of these systems requires the use of on-board software controlling the piston actuation motor. The motor can also be controlled by means of a wireless remote control. The problems encountered in these systems are mainly their production cost, complexity of use, breakdown risks and battery discharge.

Another, simpler, portable medical subcutaneous infusion pump as described in patent US20090326455 makes it possible to deliver the medicinal product by pressing simultaneously on two buttons situated on either side of the device. The dose administered corresponds to the displacement of a micro-piston expelling the medicinal product contained in a pump chamber, which is recharged when the two buttons are released. The problems encountered in this system are mainly the very large number of parts required to produce it, making the production cost high, the impossibility of automatically delivering the medicinal product continuously and the difficulty of making the system compact.

Another portable medical subcutaneous infusion pump as described in patent US20110306929 makes it possible to automatically deliver the medicinal product continuously and in doses, by manually actuating a press-button. Continuous dosing is achieved by releasing a spring pressurising a hydraulic chamber linked to the medicinal product reservoir, the flow rate from which is regulated by a calibrated restriction. The problems encountered in this system are mainly the very large number of parts required to produce it, leading to a large production cost, and the difficulty of producing a compact system.

### DESCRIPTION OF THE INVENTION

The invention relates to a miniature portable pump with simple mechanical actuation, for injecting fluid by manual dosing and/or continuously and automatically. The high-performance pump consists of a small number of parts with very low production cost, for injecting medication, preferably subcutaneously. This invention remedies the problems set out above and permits simplified development of very large-scale production of pumps, preferably inexpensive, disposable pumps made of plastics.

The pump comprises a rounded case preferably formed as two shells, which accommodate inside them a curved piston that is displaced by rotation inside a curved cylinder, one of the ends of which is linked to a cannula allowing a medicinal product to be injected subcutaneously. A manual actuator positioned in a preferably circular opening in one of the shells of the case allows the piston to be displaced by means of a series of gear wheels, forming a reduction gear, connecting the actuator to a curved rack situated on the arm of the piston. Dosing takes place by turning the actuator by a predefined angle segment corresponding to an angle of rotation of the piston of a suitable size to deliver a precise dose. An actuator retaining clip allows the actuator to be rotated securely in just one direction by the angle segment correspond [sic] to the dose to be delivered. An optional rotary, regulated, mechanical movement comprising a compressed spring is also connected to the reduction gear so as to cause the gear wheels to rotate continuously through the release of the spring, which has the effect of automatically delivering the medicinal product continuously at a predefined flow rate. A mechanism for adjusting the rotational speed of the gear wheel can optionally be added to the mechanical movement so as to achieve a variable continuous flow rate. The reduction gear comprises an element supporting the drive pinion of the piston, which is deformed in a controlled manner when the pressure in the reservoir rises beyond a predefined threshold so as to allow the drive pinion of the piston to be displaced and become lodged in a stop notch situated on one of the shells of the case. If the drive pinion of the piston becomes locked in the stop notch then the reduction gear and the actuator are also locked, which allows the user to see that a blockage has been detected.

The pump is particularly well suited to low-cost production, given that it is formed mainly of parts made of easily injectable plastics and assembled automatically. The invention also relates to a simple method for manufacturing the cylinder.

### DESCRIPTION OF DRAWINGS

It will be easier to understand the present invention by reading the description of the examples, which are given purely for information and in no way restrictive, referring to the accompanying drawings in which:

FIG. 1 is an overall view of the invention

FIG. 2 is a view of the cylinder

FIG. 3 is a view of the piston/cylinder/seal assembly.

FIG. 4 is a view of the lower shell of the case accommodating the cylinder and the piston

FIG. 5 is a side view of the lower shell of the case with the manual actuator and the reduction gear

FIG. 6 is a side view of the invention

FIG. 6a is a longitudinal section along the line A-A in FIG. 6

FIG. 6b is a longitudinal section along the line B-B in FIG. 6

FIG. 7 is a plan view of the invention

FIG. 7a is a longitudinal section along the line A-A in FIG. 7

FIG. 8 is a plan view of the lower shell of the case with the manual actuator and the reduction gear

FIG. 9 is a side view of the lower shell of the case with the manual actuator and the reduction gear without the drive pinion of the piston

FIG. 10 is a view from below of the upper shell of the case with the mechanical actuator and the reduction gear without the drive pinion of the piston

FIG. 11 is a view from below of the upper shell of the case with the mechanical actuator and the reduction gear

FIG. 12 is an exploded view of the elements forming the actuator.

FIG. 13 is a view of the support plate of the reduction gear

FIG. 14 is a view of the element supporting the drive pinion of the piston

FIG. 15 is a view of a second variant of the invention without the upper shell

FIG. 16a is a view of an impression tray of the injection mould for the cylinder with a bowed core in the cylinder ejection position

FIG. 16b is a view of an impression tray of the injection mould for the cylinder with a bowed core in the cylinder injection position

FIG. 16b [sic] is the view of an impression tray of the injection mould for the cylinder with a bowed core in the injection position

FIG. 16d is the view of an impression tray of the injection mould for the cylinder with a bowed core in the ejection position with the cylinder in the injection cavity

The diagram 17 illustrates the phases of a process for manufacturing the bowed cylinder

FIG. 18 is an overall view of a third variant of the invention with accessory parts for filling and inserting the cannula.

FIG. 18a is a side view of the third variant of the invention with the accessories.

FIG. 18b is a plan view of the third variant of the invention with the accessories

FIG. 18c is a longitudinal section along the line C-C in FIG. 18b

FIG. 19a is a side view of the cannula insertion device.

FIG. 19b is a plan view of the cannula insertion device.

FIG. 19c is a longitudinal section along the line A-A in FIG. 19a

FIG. 19d is a longitudinal section along the line B-B in FIG. 19b

FIG. 19e is an exploded view of the cannula insertion device.

FIG. 19f is a view from below of the operating handle of the cannula insertion device.

FIG. 19g is a front view of the rotor of the cannula insertion device.

FIG. 20 is an overall view of the third variant of the invention

FIG. 20a is a side view of the third variant of the invention

FIG. 20b is a longitudinal section along the line A-A in FIG. 20a

FIG. 20c is a longitudinal section along the line B-B in FIG. 20a

FIG. 20d is a plan view of the third variant of the invention without the upper shell.

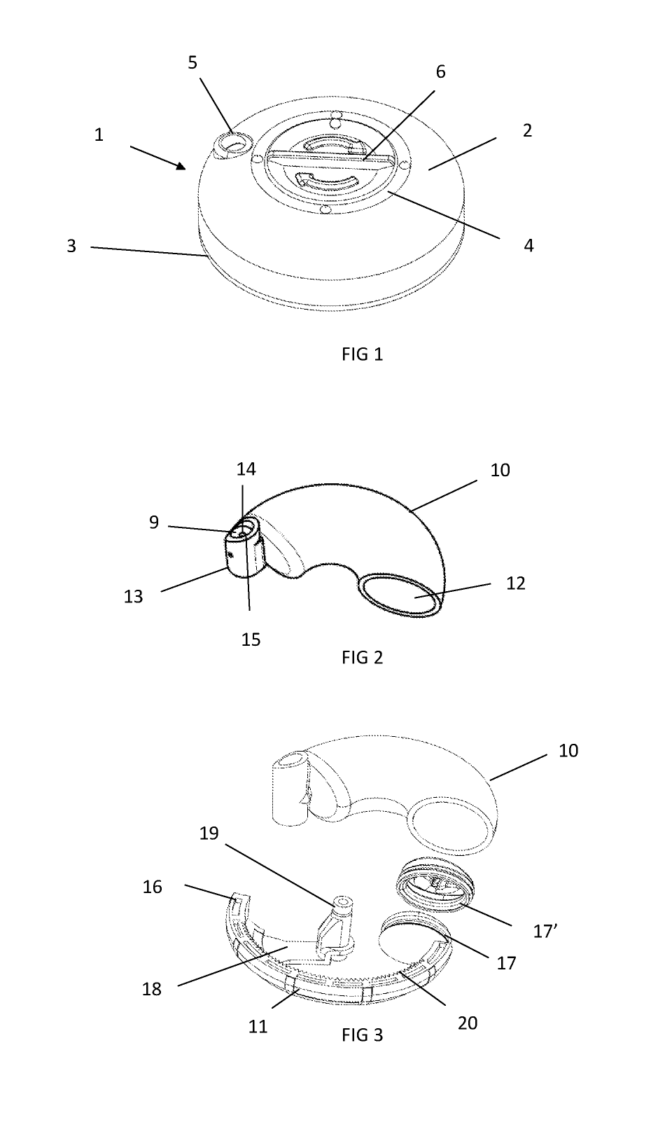

According to FIG. 1 the pump (1) comprises a lower shell (3) and an upper shell (2) forming a preferably sealed case. The lower shell comprises an adhesive support intended to keep the pump adhered to the skin of the patient. The upper shell (2) comprises a preferably circular opening inside which a mechanical manual actuator (4) is positioned, preferably having a rotational direction indicator and a gripping element (6) for the fingers. This gripping element (6) can optionally release the manual actuator (4) when gripped by the fingers. According to FIG. 2, a reservoir in the shape of a bowed cylinder (10) with the form of a portion of a torus comprises an opening (12) at one of its ends, preferably oval in cross-section, and a support (13) at its other end. The support (13) comprises a transverse opening (15) accommodating a septum (9) having a transverse channel (14) for accommodating a removable cannula (not illustrated). The septum (9) comprises a second inner channel (not illustrated) linking the inner cavity of the bowed cylinder (10) to the transverse channel (14) allowing the cannula to be linked to the fluid reservoir when the subcutaneous cannula is positioned on the patient. According to FIG. 3, a bowed piston (11) comprises an arm (16) preferably having a rack (20) on its inner face, a support (18) linked to the arm (16) and a supporting element (19) serving to fix the piston (11) at the centre of one of the shells (2, 3), and a piston head (17) that can receive a sealing element (17′) preferably in the form of an added seal, or one or more O-rings. The piston (11) is adapted so that it can be displaced within the bowed cylinder (10) while rotating about the central axis of the case.

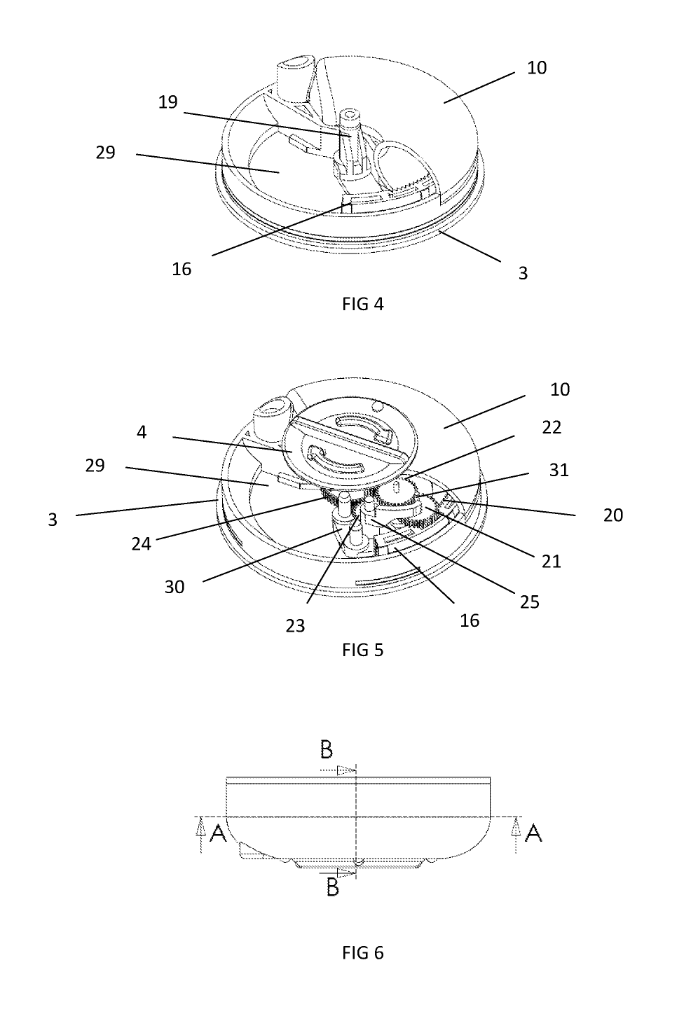

According to FIG. 4, the piston (11) and the bowed cylinder (10) are positioned in the lower shell (3) and the supporting element (19) is connected to the centre of the lower shell (3), preferably on a bearing.

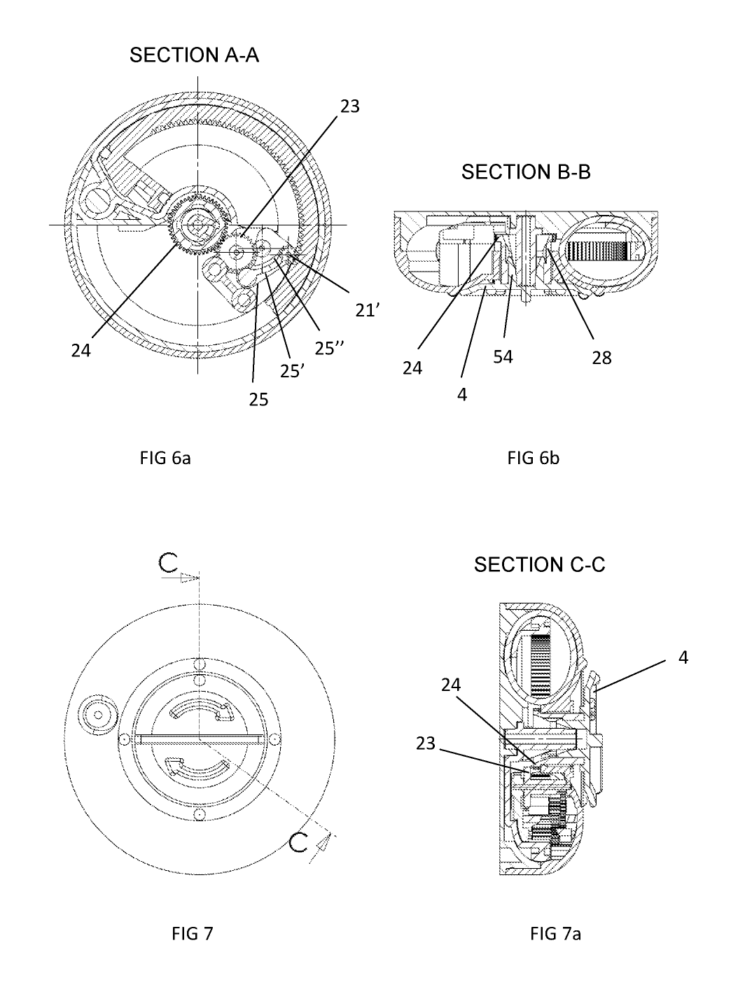

According to FIGS. 5, 6a and 6b, a manual actuator (4), preferably rotary, is positioned preferably vertically and parallel to the axis of rotation of the piston (11) in the centre of the case. The manual actuator (4) allows a drive pinion (24) situated on the lower portion of the manual actuator (4) to be actuated, inside the case. The upper bearing (not illustrated) of the supporting element (19) is situated at the centre of the manual actuator (4) opposite the bearing of the lower shell (3). A plate (30), positioned inside the cavity (29) opposite the bowed cylinder (10) comprises a reduction gear linked to the manual actuator (4) and the rack of the piston (20). The reduction gear preferably comprises a pinion (23) driven by the drive pinion (24), a pinion (22) driven by the pinion (23) and an outlet pinion (21) driven by the pinion (22). The outlet pinion (21) drives the rack (20) of the piston (11) when the manual actuator (4) rotates by means of the pinions (22, 23 and 24). A supporting element (25) is positioned on the plate so as to bear on the axle (31) of the outlet pinion (21). The supporting element (25) preferably comprises two parallel arms (25′, 25″) preferably squeezing the axle (31) of the drive pinion (21) at both ends, so as it keep it bearing against the lower shell (3).

According to FIGS. 6b, 7a and 11, the manual actuator (4) is displaced vertically between a low position putting the drive pinion (24) into contact with the pinion (23) and a high position releasing the drive pinion (24) from the pinion (23). The high position of the manual actuator (4) allows the piston (11) to be displaced freely when the reservoir is filled via the transverse channel (14) without turning the manual actuator (4). The high position of the manual reservoir [sic] (4) is used preferably as a means of checking that the pump has not been used when it is taken out of its package. A detachable locking element is preferably positioned between the manual actuator (4) and the case, to hold the manual actuator (4) in the high position when the pump is being transported and handled before use. When moved to the low position, the manual actuator (4) is preferably clipped in the bearing state, and displaces the drive pinion (24) so as to put it in contact with the pinion (23). In the low position, the manual actuator (4) is preferably locked by two clips (56), positioned at 180° on the support (28), bearing on the upper shell (2) so as to be held in this position in order to secure the piston (11) to the manual actuator (4) after the reservoir has been filled.

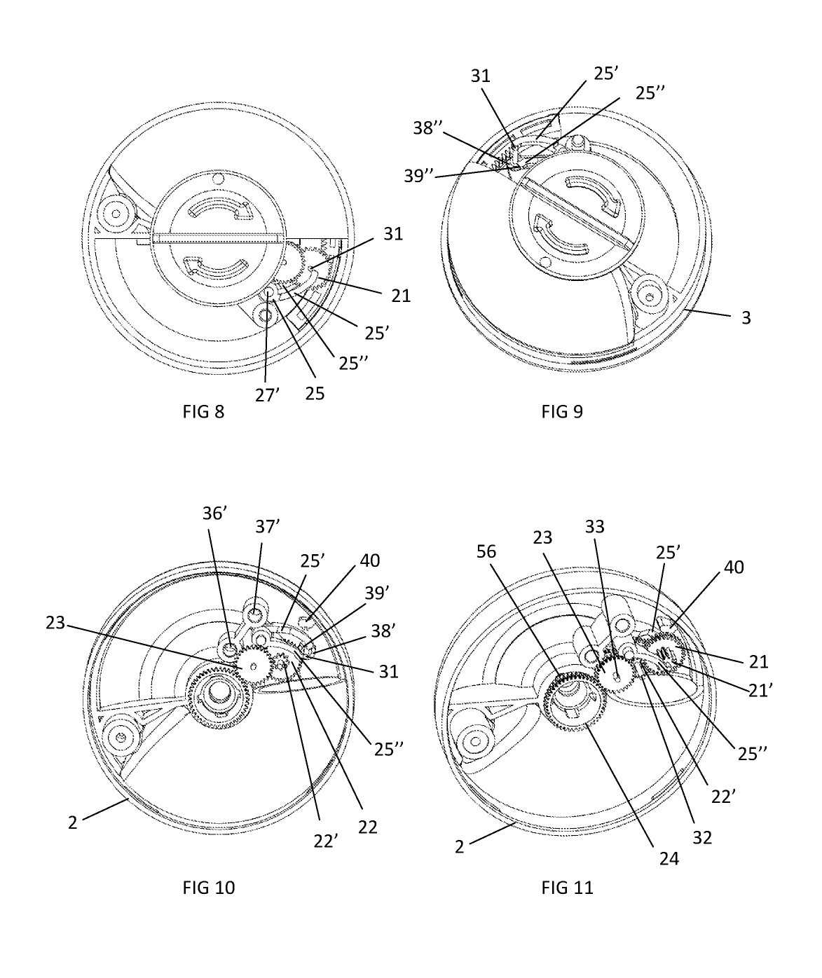

According to FIGS. 8, 9, 10, 11 and 14, the supporting element (25) is fastened to a pin (27′) in the upper shell (2) and to another pin (27″) in the bearing (35″) of the plate (30). The branches (25′, 25″) of the supporting element (25) squeezes the pin (31) of the outlet pinion (21) against the shells (2, 3) of the case respectively against the supports (38′ 38″) accommodating the pin (31) and preferably each having a 90° opening (39′, 39″). The openings (39′, 39″) are positioned asymmetrically opposite each other. The openings (39′, 39′[sic]) are positioned so as to allow the pin (31) of the outlet pinion (21) to be displaced preferably along the openings (39′, 39″) in the direction of the pin (27′) for securing the supporting element (25) when the force applied to the manual actuator (4) increases, beyond a threshold, through the resistance exerted by the rack (20) of the piston (11) on the outlet pinion (21) resulting from increased pressure in the reservoir, or from the piston (11) being locked. The branches (25′, 25″) of the supporting element (25) are constructed so as to move apart when the effort on the pin (31) exceeds a threshold corresponding to a predefined force for the resistance of the rack (20) on the outlet pinion (21). Profiles (26′, 26″) positioned on the branches (25′, 25″) ensure that the pin (31) is held in its axis of rotation when the resistance force of the rack (20) on the outlet pinion (21) is below the predefined threshold corresponding to normal operation of the pump. The displacement of the pin (31) is guided by the supports (38′, 38″) so as to orient the outlet pinion (21) in the direction of a stop notch (40), preferably positioned inside the upper shell (2), and lock it. The gear wheel and the manual actuator (4) lock when the outlet pinion (21) is stopped by the stop notch (40), which prevents the user from actuating the manual actuator (4) and thus warns him or her that the predefined maximum threshold of force has been exceeded. This mechanism for detecting a threshold of the bearing force on the piston is particularly useful for detecting a blockage or malfunction of the pump.

According to FIGS. 11 and 13, the plate (30) comprises at least one supporting element (36, 37) that becomes fastened to at least one support (36′, 37′) of the upper shell (2). It is essential for the plate (30) to be fixed so as to leave a space between the plate (30) and the lower shell (3) so that the support (18) of the piston (11) can be displaced freely during the movement of the piston (11). The plate (30) comprises retaining holes (32″, 33″) accommodating respectively an end of each axle of the pinions (32, 33). The shell (2) comprises retaining holes, not illustrated, accommodating the other ends of the pinion pins (32, 33) so as to form, together with the plate (3), a rigid support for the gear wheel ensuring that the piston is driven precisely.

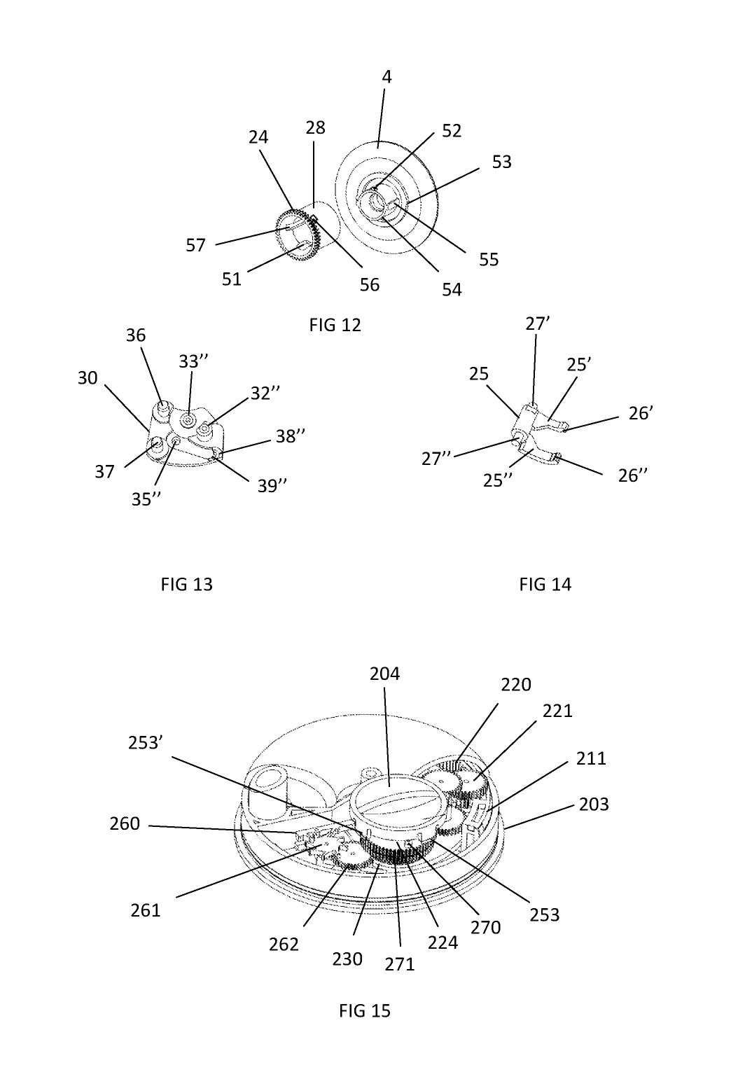

According to FIGS. 6b and 12, the manual actuator (4) comprises securing elements (52, 53) such as clips, preferably situated on the side opposite the side where the user actuates the manual actuator (4). The securing element (52) is preferably situated on a support (54) preferably positioned on the side opposite the side where the user actuates the manual actuator (4). A support (28) situated on the drive pinion (24) is connected to a support (54) comprising at least one retaining element (55), that becomes lodged in a slot (57), so as to form a rigid assembly allowing the movement of the manual actuator (4) to be transmitted to the drive pinion (24). The manual actuator (4) is held on the drive pinion (24) by at least one securing element (52), preferably in the form of a clip, which becomes lodged in an opening (51) preferably situated on the support (28). The securing element (53), preferably in the form of a clip, comes into contact with one of the shells of the case so as to produce a non-return stop notch, when it arrives at a housing situated on one of the shells of the case, corresponding to a dose to be delivered when the user actuates the manual actuator (4). The shell of the case in contact with the securing element (53) can comprise several housing so as to deliver several successive doses during a 360° rotation of the manual actuator (4).

### Second Variant

According to FIG. 15 the manual actuator (204) is positioned eccentrically relative to the axis of rotation of the piston (211). The gear wheel connecting the manual actuator (204) to the rack (220) comprises a series of pinions arranged so as to drive the piston (211) via the manual actuator (204) and, simultaneously or alternately, an automatic mechanical movement. The automatic mechanical movement comprises a power source, preferably in the form of a compressed torsion spring, serving as an autonomous actuator of the gear wheel driving the piston (211). The automatic mechanical movement preferably comprises a regulator (260, 261), acting as a time base, controlling the actuation speed of the gear wheel and thus displacing the piston (211) so as to deliver a preferably constant flow. The time regulator comprises a set of parts, not illustrated, serving to implement the automatic mechanical movement with a small number of parts.

When the manual actuator (204) is pressed, the drive pinion (224) of the automatic mechanical movement is disengaged and the pinion (271) is engaged so that turning the manual actuator (204) delivers a defined volume. The rotational movement of the manual actuator (204) is preferably performed after the pressing movement, to prevent the drive pinion of the automatic mechanical movement and that of the manual actuator from turning at the same time.

The manual actuator (204) is locked in rotation, when in the rest position, by at least one lug (270) situated on the drive pinion (271) that becomes lodged in the upper shell. Securing elements (253, 253′), preferably in the form of clips, come into contact with one of the shells of the case so as to produce a unidirectional non-return stop notch, when arriving at a housing situated on one of the shells of the case, corresponding to a dose to be delivered when the user actuates the manual actuator (204).

A flexible return element, preferably in the form of a spring, is positioned under the manual actuator (204) so as to reposition the manual actuator (204) in the rest position when it is not being actuated.

### Manufacturing Method

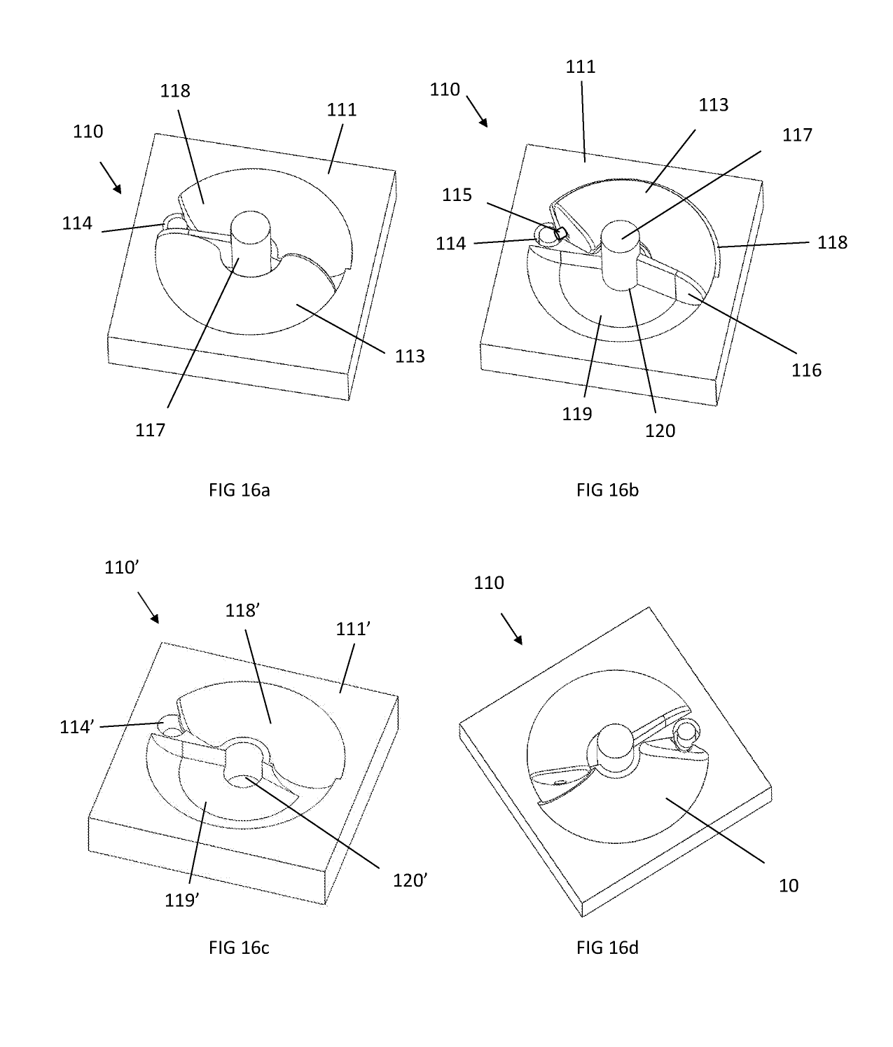

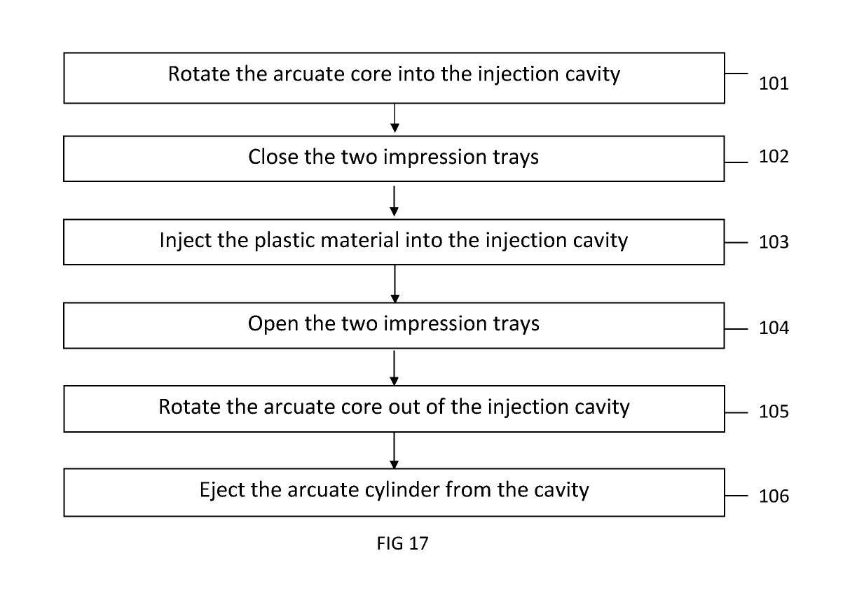

According to FIGS. 16a to 16d, an injection mould (110) comprises two impression trays (111, 111′) each having a partly toroidal cavity (118, 118′) forming, when joined, a portion of a torus corresponding to all or part of the outer surface of the bowed cylinder (10). The two impression trays (111, 111′) each comprise a partly toroidal cavity (119, 119′) that can accommodate a partly toroidal core (113), the outer surface of which corresponds to all or part of the inner surface of the bowed cylinder (10). The partly toroidal core (113) comprises, at one of its ends, a radial support (116) connected to a pin (117) that becomes lodged in the guide holes (120, 120′) situated respectively in the impression trays (111, 111′). Openings (114, 114′) preferably positioned respectively at the ends of the cavities (118, 118′) form the negative of the shape of the support (13) of the bowed cylinder (10). The partly toroidal core (113) comprises at one of its ends an element (115) in contact with at least one of the openings (114, 114′), the outer surface of which corresponds to all or part of the inner surface of the internal channel (not illustrated) linking the internal cavity to the transverse channel (14) of the bowed cylinder (10).

The partly toroidal core (113) can be actuated by any kind of mechanical, electromechanical, hydraulic or magnetic actuator so as to rotate between a retracted position when the partly toroidal core (113) is located in the cavities (119, 119′) and an injection position when the partly toroidal core (113) is located in the cavities (118, 118′). When the partly toroidal core (113) is in the injection position and the two impression trays (111, 111′) are joined together, the mould is sealed so that the material can be injected into the cavities (114, 114′, 118, 118′) forming the negative of the cylinder (10). As soon as the injection of material is finished, the material cools and hardens so as to form the cylinder (10). The partly toroidal core (113) can then be rotated into a retracted position so that the bowed cylinder (10) can be extracted when the two impression trays (111, 111′) are separated when the mould is opened. Ejectors in contact with the bowed cylinder (10) allow the bowed cylinder (10) to be ejected automatically. The element (115) can be used as a holding support with a spindle passing into at least one of the cavities (114, 114′) so as to hold the core in position during the injection phase. The mould (110) described above can have several similar impression trays so as to produce several bowed cylinders during the injection phase for the mass production of inexpensive cylinders.

According to FIG. 17, the method for manufacturing the bowed cylinder (10) preferably comprises a phase (101) consisting in rotating the partly toroidal or bowed core (113) in the injection cavity, then a phase (102) of assembling the two impression trays (111, 111′) followed by a phase (103) of injecting material, preferably thermoplastic, into the injection cavity, followed by a phase (104) of separating the two impression trays then a phase (105) of rotating the bowed core out of the injection cavity into a retracted position and ending in a phase (106) of ejecting the bowed cylinder (10). The phases (101) and (102) can be inverted, as can the phases (104) and (105). The injection phase (103) is preferably followed by a short cooling phase depending on the type of material injected.

### Third Variant

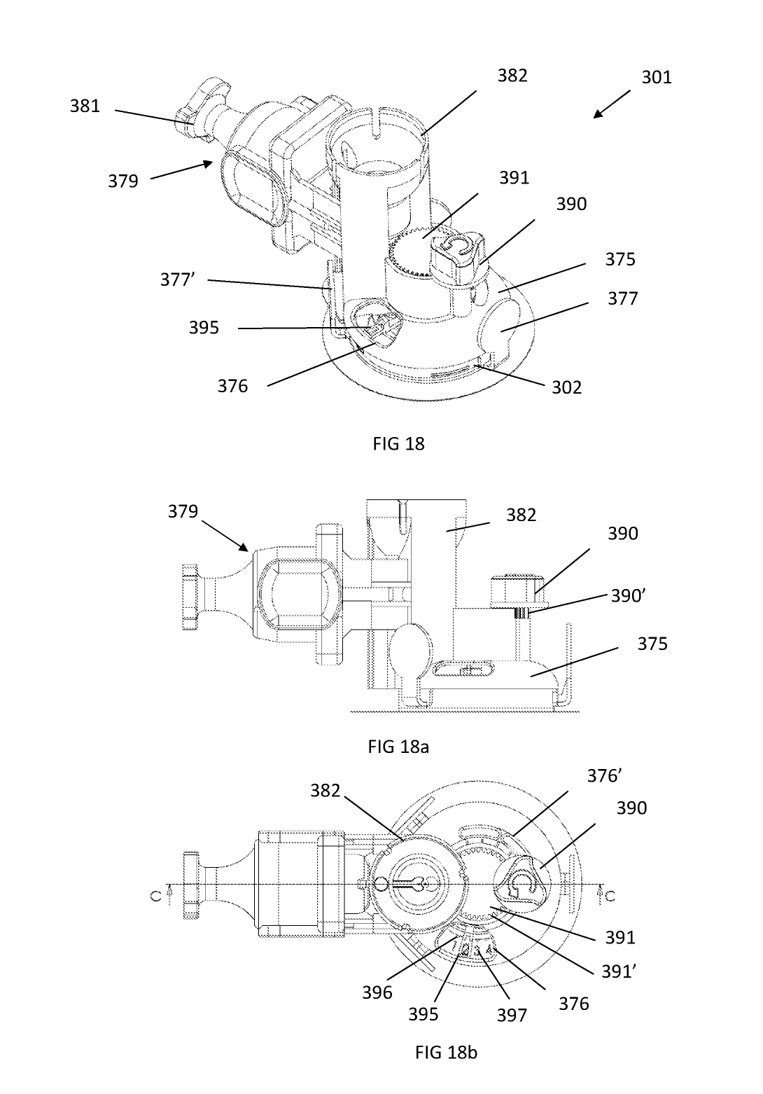

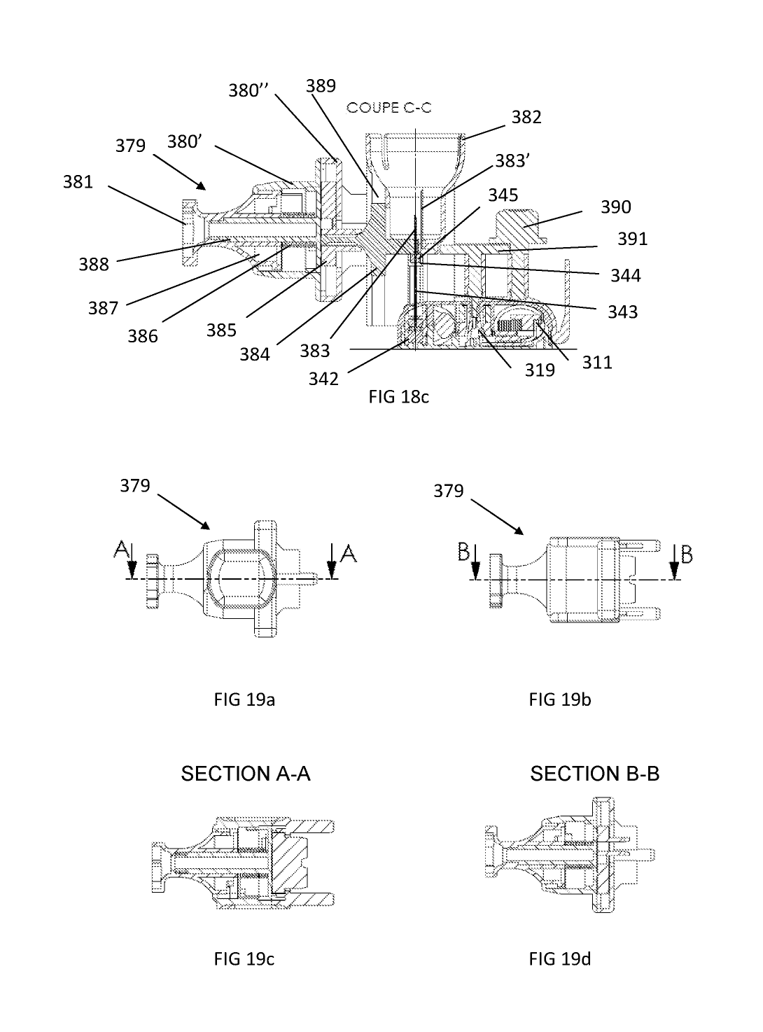

According to FIGS. 18 to 18c, the pump (301) accommodates on its upper shell (302) a support (375) preferably comprising a connector (382) intended to accommodate a vial or any other container for the fluid to be pumped, an insertion device (379) intended to insert the cannula into the patient, a thumbwheel (390) intended to displace the piston of the pump, and securing elements (377, 377′) intended to hold the support (375) on the pump (301). The support (375) preferably comprises an opening (376) intended for accessing a regulation element (395) situated on the shell (302). An actuator (391) is preferably positioned parallel to the axis of rotation of the piston so as to become linked to the supporting element (319) for the piston. A pinion (391′) situated on the actuator (391) is in contact with a pinion (390′) situated on the thumbwheel (390) so as preferably to form a reduction gear actuating the piston when the thumbwheel (390) is turned. A needle (383) situated in the connector (382) is linked, on one side, to the fluid contained in a vial positioned upside down in the connector (382) and, on the other side, is lodged in a septum (342) having a channel allowing the end of the needle to be linked to the pump chamber formed by the reservoir and the piston. Thus, when the thumbwheel (390) turns, the actuator (391) displaces the piston, which has the effect of sucking the fluid contained in the vial into the pump chamber. In this way the user can manually fill the reservoir with the desired volume. By turning the thumbwheel (390) in the other direction it is possible to empty the reservoir. The actuator (391) is preferably situated in the centre of the case so as to become engaged directly with the piston (311), to produce a simple, accurate filling mechanism with a minimum of parts. The reduction gear formed between the thumbwheel (390) and the actuator (391) is important when the cross-section of the needle is small and/or when the fluid is viscous. Indeed, if the piston is displaced too fast it would have the consequence of creating a partial vacuum that could generate air bubbles.

A needle holder (384), fixed to the needle (383), is positioned in a housing (389) situated in the connector (382) such that it can be displaced within the housing (389).

The device (379) for inserting the subcutaneous cannula is preferably removable, so that it can be detached from the connector (382) and/or support (375 and thus be reusable. The thumbwheel (390) and the actuator (391) can optionally form an assembly for actuating the piston (not illustrated) that can be detached from the support (375) so as to be reusable. The support (375) and the connector (382) are preferably disposable. It is thus possible to create different combinations of reusable and disposable elements. The support (375) and the connector (382) can be arranged so as to form a single part.

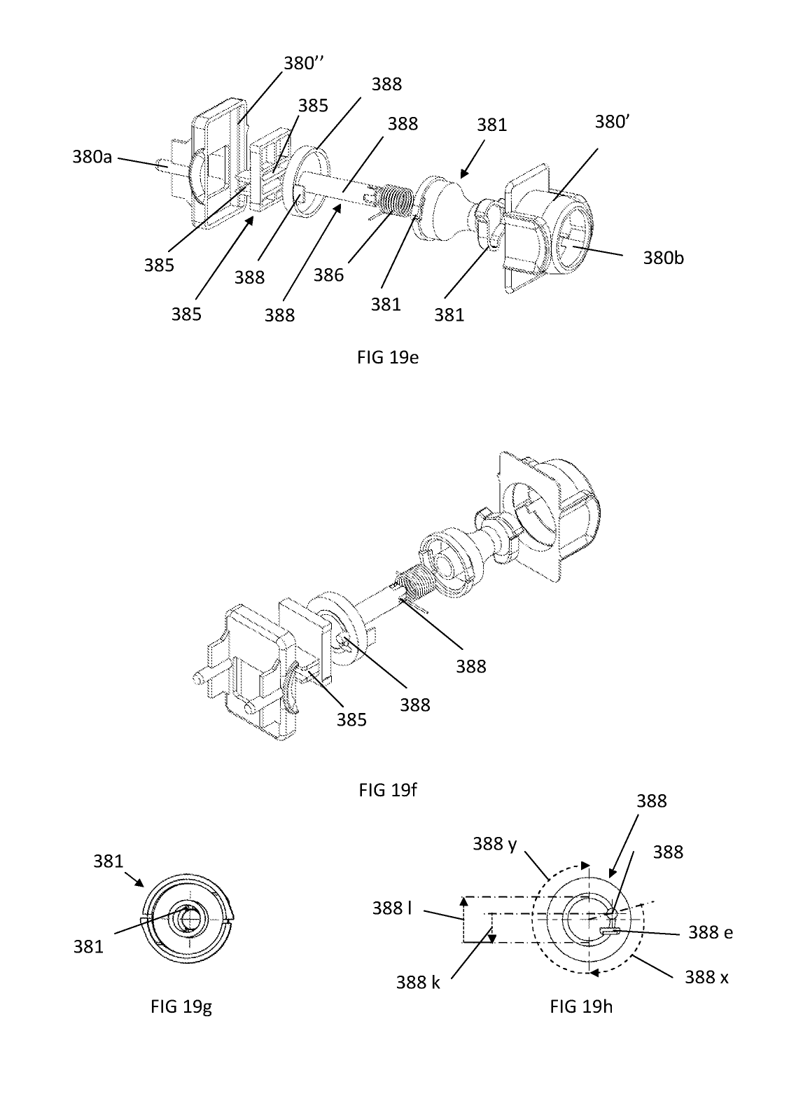

According to FIGS. 18c and 19a to 19h, the needle holder (384) is linked to an actuation element (385) situated in the insertion device (379) so that the needle (383) can be displaced when the actuation element (385) is displaced.

The insertion device (379) is preferably composed of two shells (380′, 380″) forming a case (380) in which a handle (381), a return element (386) or spring, a rotor (388) and an actuation element (385) are positioned, preferably coaxially. The device (379) for inserting the cannula (343) is armed by pushing and turning the handle (381) in the direction of the rotor (388), which is then secured to and turns with the handle (381) by at least one groove (381c) becoming engaged with at least one seat (388d) and at the same time as the return element (386) is forced, by rotation, between the seat (388e) and the inside of the shell (380′) into an orifice (380a). The handle (381) is held in the shell (380′) by means of unidirectional non-return clips (381b), so as to keep the return element (386) tensioned. The lug (388c) of the rotor (388) becomes lodged in the slot (385a) of the actuation element (385) so as to convert the rotation of the rotor (388) into a linear displacement of the actuation element (385) serving to actuate the movement of the needle (383) by driving the needle holder (384) linked to the actuation element (385). The cannula insertion device (379) is triggered by pulling the handle (381) in the opposite direction to the rotor (388) so as to release the slot (381c) from the seat (388d), which has the effect of decoupling the rotor (388) from the handle (381). The rotor (388) is then driven in rotation by the return elements (386) transmitting, via the actuation element (385), a vertical back-and-forth movement (388k, 388l) of the needle holder (384) according to the angular portions (388x, 388y) so as to displace the needle (383) towards the skin of the patient and pierce it. Once it reaches the low position, the needle (383) rises and the cannula (343) positioned around the needle (383) remains fixed in the low position on the shell (302) by a supporting element (344) attached to the cannula (343), which becomes lodged on the shell (302).

A profile (381a) situated at the outer end of the handle is preferably non-circular in shape so as to have an effective grip.

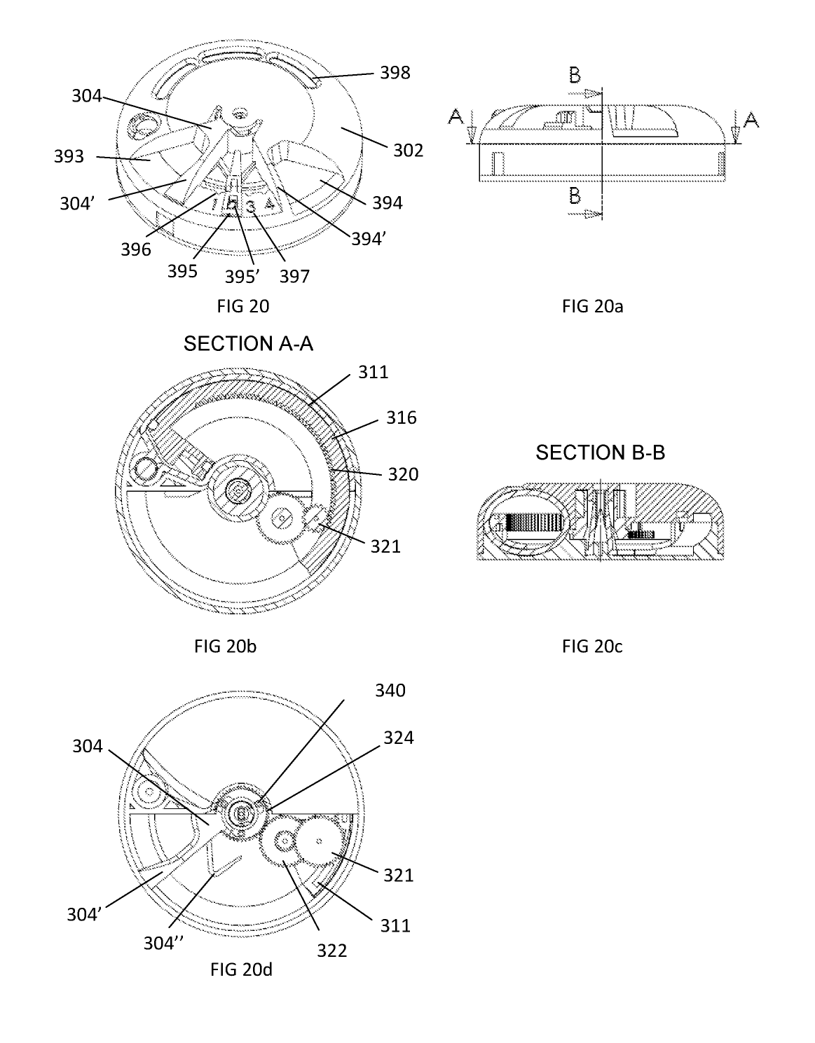

According to FIGS. 20 to 20d, the upper shell (302) preferably comprises an opening (398) allowing the reservoir to be seen inside the case, and an opening (394) or recess, preferably adjacent to an opening (393) separated from the opening (394) by a support element (394′). An actuator (304) comprising an arm (304′) or lug is preferably positioned in the opening (393) and held between the two shells (302, 303) of the case so that it can be displaced or pivoted within the opening (393) when the user presses on the arm (304′). The actuator (304) is coupled to a drive pinion (324) by means of a free wheel formed by at least one roller (340) positioned in at least one housing (340′) in order to actuate the drive pinion (324) unidirectionally. The drive pinion (324) is linked to the pinion (322), which is itself linked to an outlet pinion (321) driving the bowed piston (311) via the rack (320), situated on the arm (316) of the piston, when the pinion (324) rotates. A stop element (395), preferably rotary, situated between the arm (304′) of the actuator (304) and the support element (394′), can be moved over the stop notches (396) situated on the shell (306). The stop element (395) preferably comprises a positioning mark (395′) coming opposite a graduation (397), preferably alphanumeric, so as to indicate the position of the corresponding stop notch. When the arm (304′) is pushed in the direction of the stop element (395) the movement of the arm (304′) is limited to the position of the stop element (395) so as to regulate the displacement of the piston by an increment corresponding to the graduation (397). The graduation is preferably numeric and corresponds to a dose unit suited to medicinal products; for example, “1” corresponds to one unit of insulin 1 U (10 ul).

A return element (304″) allows the arm (304′) to be returned to the rest position before the system is actuated again.

In this way an adjustable dose can be delivered by squeezing the arm (304′) and the support element (394′) with the fingers. The system can therefore be actuated reliably and rapidly with one hand.

### Variants

The pump can be filled manually by pushing the fluid into the reservoir, for example by means of a syringe. The pump can also be filled by connecting the pump reservoir to a reservoir containing the fluid and by turning the manual actuator so as to displace the piston in the direction of the bottom dead centre. The pump can be adapted so as to use a prefilled reservoir. The pump can be adapted so as to incorporate a second septum communicating with the reservoir so that the reservoir can be filled or emptied by means of a needle passing through the septum.

The manual actuator or any of the pinions can be adapted so as to rotate in one direction only.

The pump can be adapted so that the manual actuator is displaced vertically into different positions making it possible, for example, to turn the manual actuator freely without driving the gear wheel or to lock the manual actuator in a position that prevents it from moving.

The pump can be adapted so as to hold the plate on the lower shell of the case.

The pump can be adapted so as to lock the outlet pinion in the stop notch and disengage the outlet pinion from the gear wheel. The manual actuator can then be actuated without displacing the piston. An indicator that the outlet pinion is locked, visible on the case, can be added to the pump to inform the user that the predefined threshold of force has been exceeded.

The pump can be adapted so as to connect the manual actuator to the drive pinion, according to several variants not illustrated, so as to obtain one or more relative positions of the manual actuator and the drive pinion, such as a position for transport, for activation or for locking the pump.

The pump can be adapted to that the free wheel linked to the manual actuator is replaced by a ratchet system.

The pump can be adapted so that the support element (394′) is in the form of a lug, an edge

The seal between the moveable portions is preferably produced using an elastomer, an overmoulded seal or any other sealing element. It is, however, possible to produce the pump without a sealing joint between the cylinder and the piston, for example by adjustment. The elements making up the pump are preferably made of plastics and are disposable. The pump can be sterilised for distribution of a foodstuff or medication, for example. The choice of materials is not, however, limited to plastics.

The shells can be assembled by gluing, by clip elements, by mechanical fastening or by ultrasonic welding.

The pump can be adapted so as to deliver the fluid to the patient by means of microneedles or any other transdermal means.

The cannula insertion device can be positioned above the case so as to have a compact assembly fitting into the palm of the hand.

The pump can be adapted so as to incorporate one or more electronic elements allowing the measurement of various operating parameters of the pump, such as temperature, humidity, pressure or force on all or part of the pump. The pump can incorporate one or more biological or biochemical parameter sensors, whether optical, acoustic or magnetic, or related to position, closure, acceleration or inclination.

The electronics can be powered by a battery, a rechargeable battery and/or an external power source provided by induction. The electronics can communicate with an external device by wireless communication according to any kind of protocol and data format such as WiFi, Bluetooth, NFC, RFID and other electromagnetic or optical signals. The electronics can incorporate a memory and a microprocessor so as to load data stored in the memory and execute calculation, measurement, formatting, updating, data recording and transfer operations autonomously or according to commands from an external device.

The pump can be adapted so as to incorporate one or more elements for controlling the spring release speed so as to achieve a base flow rate that is preprogrammed or remotely controllable/programmable. All or part of an element controlling the spring release speed can be actuated mechanically or electromechanically by the electronics or an external device directly supplying the power required for the movement or change of state of the element controlling the spring release speed.

The electronics can transmit and receive, with an external device, data concerning its own operation such as the state of the battery, its components or sensors, or any other information on pump control, malfunction and warnings.

Although the pump is mainly intended to be actuated without a motor, it can be adapted so as to incorporate a motor actuating all or part of the mechanical drive of the piston. The electronics can control and supervise the motor so as to deliver doses of fluid according to commands that are preprogrammed or transmitted by an external device. The motor, electronics and battery are optionally built into the pump so as to form a single-use pump with no reusable portions.

The pump can be adapted so as to incorporate one or more electronic dose-regulation elements so as to control the frequency of administration of manual doses and prevent the patient from actuating the manual actuator before a predefined period or one that can be programmed into the electronics.

The pump can be adapted so as to incorporate one or more dose-metering elements so as to inform the patient about the number of doses delivered and remaining. The case can be adapted so that the position of the piston in the cylinder can be seen, while incorporating a visual positioning mark such as a graduation.

Although several embodiments of the invention are described, there are other variants that are not presented. The scope of the invention is therefore not limited to the embodiments described above.

1. A portable pump for injecting fluid into a patient, comprising a arcuate piston, moving within a arcuate cylinder, actuated by an actuator without a motor, characterised by a case comprising at least an upper shell having at least one opening.

2. (canceled)

3. The pump according to claim 1, the actuator of which is rotary.

4. (canceled)

5. The pump according to claim 1, the actuator of which comprises a pinion.

6. (canceled)

7. The pump according to claim 1, comprising a stop element for the actuator.

8. The pump according to claim 1, comprising a return element for the actuator.

9. The pump according to claim 7, wherein the stop element for the actuator is moveable.

10. The pump according to claim 5, wherein the pinion of the actuator drives a gear actuating the arcuate piston.

11. The pump according to claim 10, wherein a pinion of the gear is in contact with a rack situated on an arm of the piston.

12. The pump according to claim 10, the gear of which serves as a reduction gear.

13. The pump according to claim 10, wherein the pinion of the actuator comprises a free-wheel or a ratchet system.

14. (canceled)

15. (canceled)

16. (canceled)

17. The pump according to claim 10, wherein the gear wheel is held by a plate fastened to at least one shell of the case.

18. The pump according to claim 1, wherein the actuator is actuated by an automatic mechanical movement.

19. (canceled)

20. (canceled)

21. (canceled)

22. The pump according to claim 10, comprising a supporting element with detection of the piston driving force threshold.

23. The pump according to claim 22, wherein one of the gear wheel pinions is locked when the piston driving force exceeds the detection threshold of the supporting element with detection of the piston force threshold.

24. The pump according to claim 1, wherein a removable support is positioned on the upper shell.

25. (canceled)

26. (canceled)

27. (canceled)

28. (canceled)

29. The pump according to claim 24, wherein a cannula insertion device is connected to the removable support.

30. (canceled)

31. The pump according to claim 29, wherein the cannula insertion device is reusable.

32. The pump according to claim 1, wherein all or some of the parts are disposable.

33. (canceled)

34. (canceled)

35. (canceled)

36. (canceled)

37. (canceled)

38. The portable pump according to claim 1, wherein the cylinder is manufactured by injection into a mould according to a method comprising at least
A phase of rotating a partly toroidal core within an injection cavity,
A phase of injecting material when the partly toroidal core is positioned in the injection cavity,
A phase of rotating the partly toroidal core in a retracted position,
A phase of ejecting the cylinder out of the mould when the core is in the retracted position.

39. (canceled)

40. (canceled)

41. (canceled)

42. The pump according to claim 29, wherein the cannula insertion device comprises a return element of which is tensioned by rotating a handle.

43. (canceled)

44. (canceled)

45. (canceled)

46. (canceled)
